(19) United States
(12) Patent Application Publication
(10) Pub. No.: US 2009/0270672 A1
(43) Pub. Date: Oct. 29, 2009

(54) Needle Cap Ejector for Radiation Shielded Syringe

(76) Inventor: Frank M. Fago, Mason, OH (US)

Correspondence Address: Mallinckrodt Inc., 675 McDonnell Boulevard, HAZELWOOD, MO, 63042, US

(21) Appl. No.: 12/162,803

(22) PCT Filed: Jan. 8, 2008
(86) PCT No.: PCT/US08/00312

§ 371 Date: Jul. 31, 2008

Related U.S. Application Data
(60) Provisional application No. 60/884,024, filed on Jan. 9, 2007.

(51) Int. Cl. A61M 36/08

(52) U.S. Cl. 600/5

(57) ABSTRACT

The invention relates to a radiation shielded syringe assembly that includes a radiopharmaceutical syringe and a radiation shield (101) disposed about at least a portion of the syringe. The radiation shield may include a needle cap ejector (155) to assist a user in removing a needle cap (125) from a needle of the syringe (e.g., to perform an injection). For instance, in some embodiments, the user may press/push the needle cap ejector to detach the needle cap from the needle of the syringe. The radiation shield and needle cap ejector may be designed such that the needle cap may be removed from the needle of the syringe while the user is shielded from undesired exposure to radiation emitted from the radiopharmaceutical within the syringe.

### FIELD OF INVENTION

The present invention relates generally to radiation shielding devices for shielding radioactive materials and more particularly to radiation shielding devices used to safely handle radiopharmaceuticals contained in syringes having attached needles.

### BACKGROUND

Nuclear medicine is a branch of medicine that uses radioactive materials (e.g., radioisotopes) for various research, diagnostic and therapeutic applications. Radiopharmacies produce various radiopharmaceuticals (i.e., radioactive pharmaceuticals) by combining one or more radioactive materials with other materials to adapt the radioactive materials for use in a particular procedure.

It is common for solutions containing radioisotopes and liquid radiopharmaceuticals to be contained in syringes at various times during preparation and use of radiopharmaceuticals. For example, aliquots of radioisotope-containing eluates (e.g., solutions containing Technetium-99 obtained from a radioisotope generator) are often drawn by syringe to prepare a dose of a particular radiopharmaceutical from that eluate. Likewise, a syringe may be used to inject a dose of a liquid radiopharmaceutical into a patient. The syringes often include needles (e.g., hypodermic needles), which may be used to pierce a septum seal of a container and/or the skin of a patient receiving a radiopharmaceutical injection. To prevent accidental needlestick injuries, the needles of the syringes are commonly covered by a protective needle cap that is releasably attached to the syringe.

Radiation exposure is also a hazard for those frequently handling syringes containing radioactive materials. Syringes containing radioactive materials are commonly placed in radiation shields to reduce radiation exposure to those handling the syringe. The radiation shields contain lead, tungsten, depleted uranium, or a similar dense material. Radiation shields commonly comprise a tubular shielding body defining a cavity that houses the barrel of the syringe. For example, the bodies of some radiation shields are sleeves (e.g., substantially circular in cross section) that extend circumferentially around the side of the syringe barrel for approximately the length thereof.

Many radiation shields are used during aspiration of the radioactive material into the syringe and/or injection of the radioactive material from the syringe. To facilitate aspiration and/or injection of the radioactive material, the needle (and possibly a relatively small portion of the barrel connecting to the needle) extends through an opening at the front end of the shielding body to the exterior of the cavity, so the tip of the needle extends to the exterior of the radiation shield and can pierce a septum sealed container or a subject's skin. Unfortunately, radiation emitted by the radioactive material in the syringe can escape through that opening. Further, users may place their hands in close proximity to the opening while removing the protective needle cap from the syringe. Thus, users may undesirably be exposed to radiation escaping the radiation shield through the opening at the front end of the shielding body.

### SUMMARY

The present invention, in certain embodiments, relates to radiation shielded syringes equipped with a needle cap ejector, as well as methods of removing a needle cap from a radiation shielded syringe. Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of features and aspects that may not be set forth below.

One aspect of the invention is directed to a radiation shielded syringe assembly that includes a syringe, a radiation shield, a needle cap, and a needle cap ejector. The syringe of the assembly includes both a barrel for containing a radioactive substance and a needle at one end of the barrel. The needle cap is releasably attached to the syringe and covers at least the tip of the needle. The radiation shield of the assembly has a cavity defined therein and at least one opening that extends into the cavity. At least a portion of the barrel of the syringe is located within the cavity of the radiation shield, and at least a portion of the syringe, including at least a tip of the needle, protrudes through the opening in the radiation shield to an exterior of the radiation shield. The needle cap ejector of the assembly may be utilized to selectively detach the needle cap from the syringe. This needle cap ejector includes an engagement member having an engagement surface located outside a zone of radiation exposure defined by all locations within an axial projection of the opening away from the radiation shield. The needle cap ejector is arranged so that the needle cap can be detached from the syringe by manual application of a detachment force to the engagement surface. For example, a person may detach the needle cap from the syringe by manually applying a force to a portion of the needle cap ejector that is located outside of the radiation shield. Because a person can apply the force needed to detach the needle cap from the needle to a structure of the assembly that is sufficiently remote from the opening in the radiation shield, the radiation shielded syringe assembly can be utilized to limit the person's exposure to radiation.

Another aspect of the invention is directed to a radiation shield for a syringe. The radiation shield has a body made of a radiation shielding material (e.g., lead, tungsten, tungsten-impregnated plastic, etc.). There is a cavity defined inside the shield for receiving at least a portion of a syringe barrel. There is also an opening defined in a front end of the radiation shield through which at least a tip of a syringe needle protrudes when a syringe is in the radiation shield. The body of the radiation shield supports a needle cap ejector that may be utilized to detach a needle cap from the syringe when a person applies a force to the needle cap ejector. The needle cap ejector may be arranged so that a person can apply the force needed to detach the needle cap at a location that is remote from the opening in the radiation shield, thereby potentially reducing unnecessary exposure to radiation.

Yet another aspect of the invention is directed to a method of using a radiation shielded syringe assembly that includes a syringe and a radiation shield disposed about at least a portion of the syringe. In this method, a force (e.g., applied by a user) is exerted on a needle cap ejector of the syringe assembly while radiation shielding material of the radiation shield is located between the ejector and a barrel of the syringe. Due to the exertion of this force, a needle cap is detached from a needle of the syringe.

Various refinements exist of the features noted above in relation to the various aspects of the present invention. Further features may also be incorporated in these various aspects as well. These refinements and additional features may exist individually or in any combination. For instance, various features discussed below in relation to one or more of the illustrated embodiments may be incorporated into any of the above-described aspects of the present invention alone or in any combination. Again, the brief summary presented above is intended only to familiarize the reader with certain aspects and contexts of the present invention without limitation to the claimed subject matter.

### BRIEF DESCRIPTIONS OF THE FIGURES

FIG. 1 is a perspective of one embodiment of a radiation shield for a radiopharmaceutical syringe;

FIG. 2 is a longitudinal section of the radiation shield;

FIG. 3 is a perspective of radiation shielded syringe assembly including the radiation shield of FIGS. 1-2 on a radiopharmaceutical containing syringe;

FIGS. 4A-4C are fragmentary longitudinal sections of the radiation shielded syringe assembly illustrating a sequence in which a needle cap is detached from the syringe using a needle cap ejector of the radiation shield;

FIG. 5 is a perspective view of another embodiment of a radiation shielded syringe assembly;

FIGS. 6A-6B are longitudinal sections of the radiation shielded syringe assembly shown in FIG. 5 illustrating a sequence in which a slide mechanism is used to detach a needle cap from the syringe;

FIG. 7 is an enlarged detail of a portion of the slide mechanism of FIGS. 6A-6B;

FIG. 8 is a perspective of another embodiment of a radiation shielded syringe assembly including a needle cap that has an integral cap ejector; and

FIGS. 9A-9B are longitudinal sections illustrating a sequence in which the needle cap shown in FIG. 8 is detached from the syringe.

Corresponding reference characters indicate corresponding parts throughout the figures.

### DETAILED DESCRIPTION OF EXEMPLARY EMBODIMENTS

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time-consuming, but would nevertheless be a routine undertaking of design, fabrication, and manufacture for those of ordinary skill having the benefit of this disclosure.

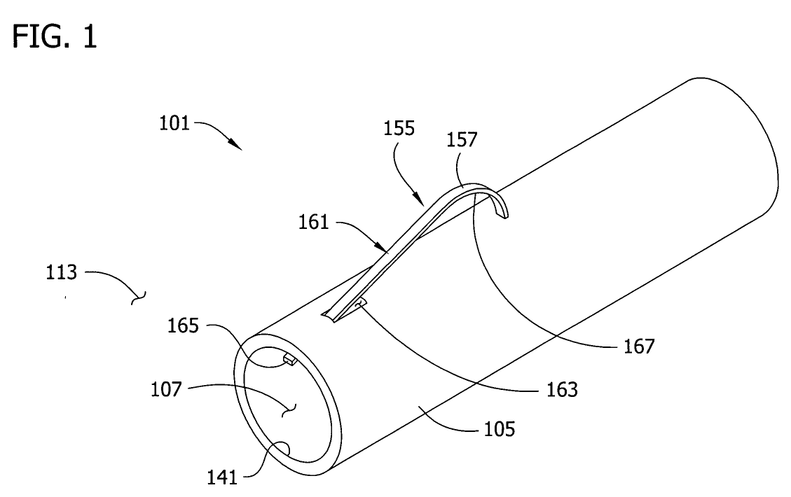

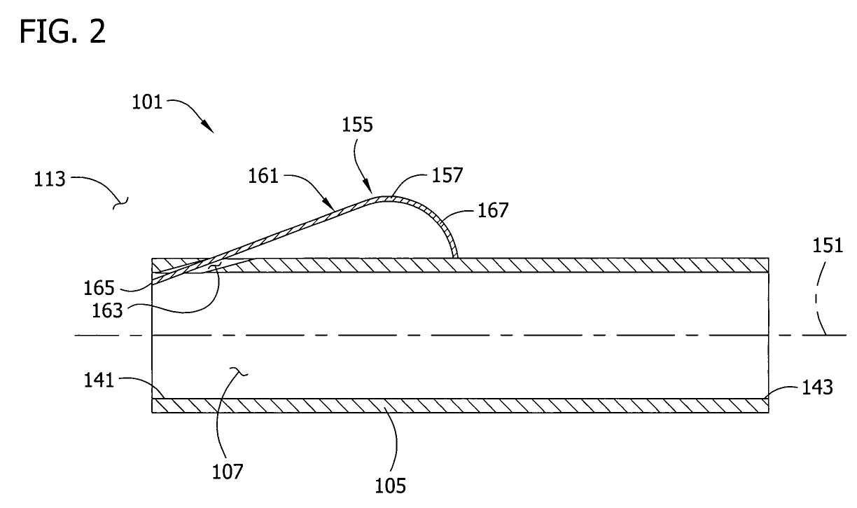

Referring now to the figures, first to FIGS. 1-3, one embodiment of a radiation shield of the present invention is generally designated 101. The radiation shield 101 is suitable for use with a syringe 103 containing one or more radioactive materials 120 (e.g., radiopharmaceuticals such as or that include radioisotope-containing eluates). The radiation shield 101 comprises a substantially tubular radiation shielding body 105 constructed to absorb radiation, for example by virtue of it including one or more of lead, depleted uranium, tungsten, or another suitably radiation dense material. The shielding body 105 has a cavity 107 defined therein for receiving at least a portion of a barrel 109 of the syringe 103 in the cavity to form the assembly 111 shown in FIGS. 34C.

The body 105 of the radiation shield 101 shown in the figures is shaped to form a sleeve extending circumferentially around the side of the syringe barrel 109 substantially along the full length of the barrel. More particularly, the body 105 may be tubular defining a generally tubular cavity 107 suitable for receiving the syringe barrel 109, which is also generally tubular in the illustrated embodiment. The body 105 is sized so that a user is able to hold the radiation shield 101 in a single hand. The body 105 is also shaped and arranged so that a users hand(s) may be shielded from radiation emitted in the cavity 107 while holding the radiation shield 101 in one or both hands. Those skilled in the art will know how to select a thickness of the body 105 to provide a desired level of radiation shielding in view of the particular material(s) making up the body and in view of the characteristics of the radioactive material(s) to be contained in the cavity 107. Further, the body 105 can be configured differently than shown without departing from the scope of the invention.

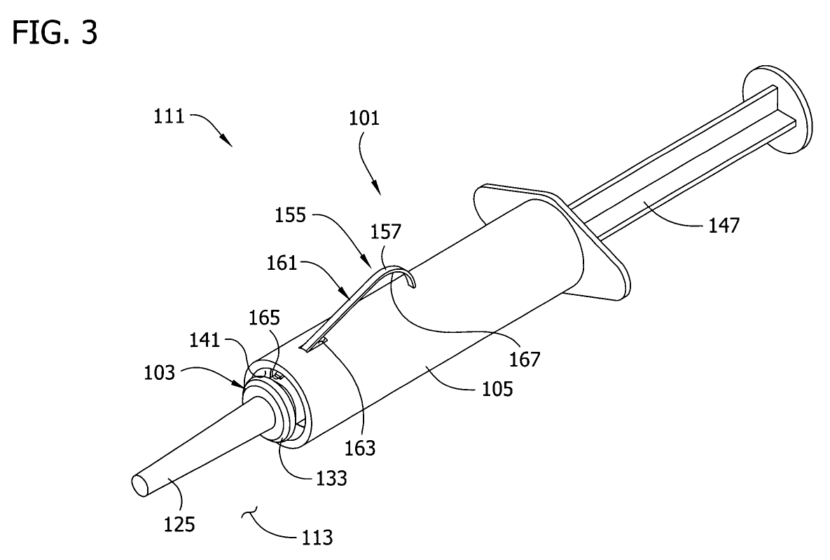

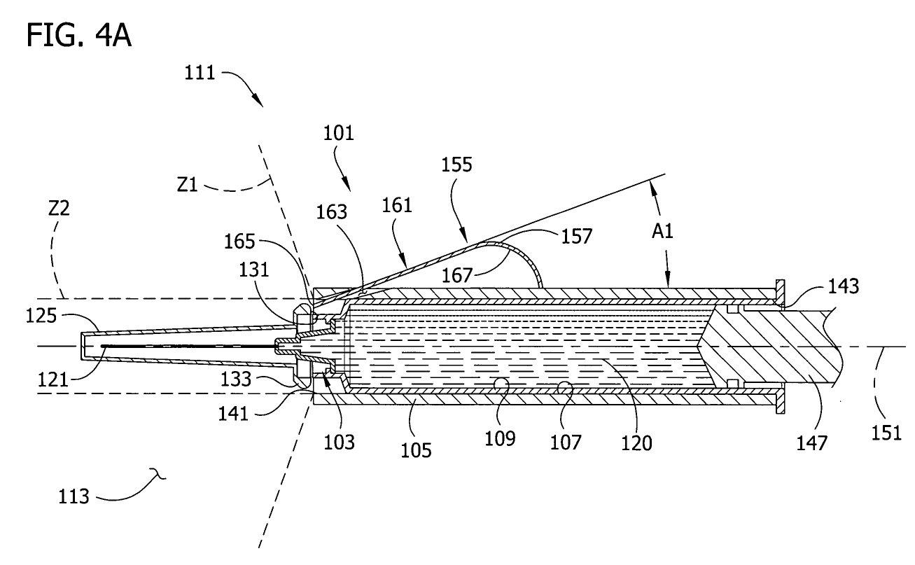

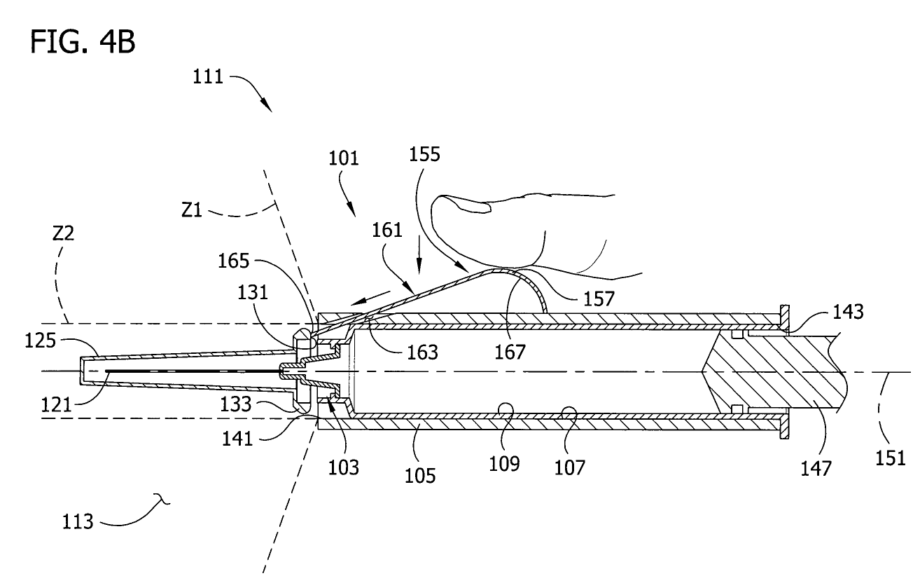

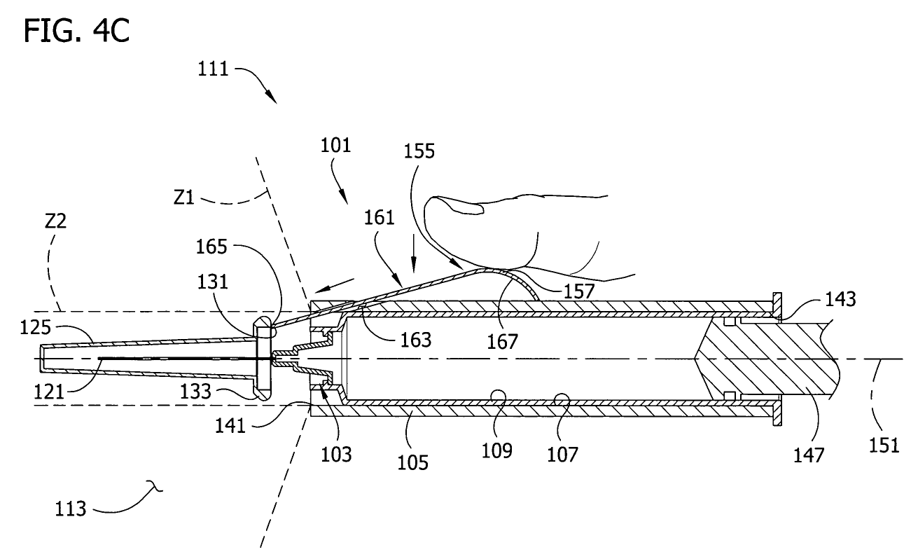

The syringe 103 shown in FIGS. 3-4C is a conventional syringe having a needle 121 (FIG. 4A) attached to the front end of the syringe barrel 109 (e.g., via a luer connection). A radioactive material 120 (e.g., a radiopharmaceutical), which is shown in FIG. 4A but omitted from the other figures, is contained in the syringe barrel 109. A protective needle cap 125 is attached to the syringe 103 (e.g., to the front of the syringe barrel 109) so that the needle cap covers at least the tip of the needle 121. The needle cap 125 shown in the figures comprises a generally funnel-shaped sheath having an open end for receiving the needle therein and closed end at the opposite end of the needle cap. There is an axially facing surface 131 at the open end of the needle cap. In the embodiment shown in the figures, the needle cap 125 has an annular flange 133 extending radially outward at its open end, resulting in a generally flat axially facing surface 131 at the open end of the needle cap. However, the flange 133 is not required for the present invention. When the needle cap 125 is placed over the needle 121 and the open end of the needle cap is attached to the syringe 103, the needle is completely enclosed within the protective sheath of the needle cap. As is known to those skilled in the art, there are various ways that a needle cap can be releasably attached to the syringe 103. The needle cap can use virtually any attachment system in which the needle cap can be released by pushing it away from the syringe and can be attached to the syringe at various locations (e.g., at the syringe barrel, at the needle, at a connecting member used to connect the needle to the syringe barrel, etc.) without departing from the scope of the invention. For example, the connection can be through an interference fit of the needle cap 125 with the syringe 103. Needle caps having different configurations than the needle cap 125 shown in the figures may be used without departing from the scope of the invention.

The body 105 of the radiation shield 101 has an opening 141 defined therein that allows the needle 121 to project from the cavity 107 at its front end for delivery of a substance into the barrel 109 of the syringe 103 through the opening (e.g., aspiration of a radiopharmaceutical into the syringe barrel through the needle) and/or for delivery of the substance contained in the barrel of the syringe to the exterior 113 of the cavity through the opening (e.g., during injection of a radiopharmaceutical using the needle). As shown in FIGS. 3-4C, the front of the syringe 103, including at least the tip of the needle 121 and possibly the front of the syringe barrel 109, protrudes through the opening 141 to the exterior 113 of the cavity 107 so that the tip of the needle can be exposed by removing the needle cap 125 from the syringe. Moreover, after the tip of the needle 121 is exposed, the needle can be used to pierce a septum seal (e.g., of a supply container) or pierce a subject's skin while the syringe 103 remains in the radiation shield 101.

In the embodiment shown in FIGS. 1-4C, the body 105 of the radiation shield 101 has another opening 143 defined therein that extends into the cavity 107 at its rear. A syringe plunger 147 slidably received in the syringe barrel 109 extends from inside the barrel to the exterior 113 of the radiation shield 101 through the rear opening 143 so that a user may access the plunger to aspirate the radioactive material into the syringe 103 and/or expel the radioactive material from the syringe. The plunger 147, or at least a portion thereof, may contain one or more radiation shielding materials (not shown) to block escape of radiation emitted in the cavity 107 through the rear opening 143.

When a syringe 103 containing a radioactive material is in the cavity 107, there is a substantially unshielded path for radiation emitted in the cavity to escape the radiation shield 101 through the front opening 141. Thus, there is an increased radiation threat adjacent the opening 141. Because radiation travels along a generally linear path, a zone Z1 that includes all points adjacent the opening having a direct line of sight (disregarding objects that are substantially transparent to the radiation) to radioactive material contained in the barrel 109 represents a zone of increased radiation threat. The zone Z1 extends away from the opening to a distance that is relatively safe because of attenuation and/or dispersion of the radiation after it has propagated that distance. The shape of the zone Z1 will depend on the shape of the opening 141 and the shape of the syringe barrel 109. In the illustrated embodiment, for example, the opening 141 is substantially circular, and the barrel 109 is substantially cylindrical resulting in the zone Z1 that has a direct line of sight to at least a portion of the syringe barrel 109 and that has a conical or frusto-conical shape aligned co-axially with the longitudinal axis 151 of the cavity 107/syringe barrel that widens as it extends away from the opening 141.

The threat of exposure to radiation is greatest at the opening 141 and along the longitudinal axis 151 of the cavity 107/syringe barrel 109 a short distance away from the opening. For example, a zone Z2 defined to include all points adjacent the opening 141 within an axial projection of the opening along the longitudinal axis 151 generally corresponds to a greater radiation threat than the edge of the zone Z1. In the illustrated embodiment, for example, locations within the zone Z2 have a direct line of sight to substantially the entire syringe barrel 109 and therefore to a substantial amount of the radioactive material therein, while the locations at the edge of zone Z1 only have a direct line of sight to a small portion of the syringe barrel 109.

As indicated in FIG. 4A, the needle cap 125 may connect to the syringe barrel 109 at the opening 141. In other words, the needle cap 125 (and the part of the needle cap that makes the connection with the syringe 103) is located within zone Z1, and more particularly within Z2, and still more particularly right at the front opening 141 of the radiation shield 101. Thus, the needle cap 125 is located in the area exposed to the most radiation.

It is understood that small amount of radioactive material may be forward of the barrel 109 in the syringe. For example, at least some residue of the radioactive fluid is likely to remain in the needle 121 after the material is aspirated into the barrel 109 through the needle. Further, some radioactive material may be in the formations connecting the needle 121 to the syringe barrel 109. It will be recognized that the quantities of material that could be positioned in the syringe 103 forward of the syringe barrel 109 are significantly smaller than the quantity that can be contained in the syringe barrel. Thus, there is substantially less radiation emitted by any radioactive material that is forward of the barrel 109 in the syringe than is emitted in the barrel. Accordingly, exposure to radiation emitted by materials in the syringe 103 forward of the syringe barrel 109 is less of a concern than exposure to the radiation emitted by the larger quantity of material in the syringe barrel.

The radiation shield 101 includes a needle cap ejector 155 operable to detach the needle cap 125 from the syringe 103 upon manual application of a detachment force to an engagement surface of an engagement member 157 of the ejector. The needle cap ejector 155 shown in FIGS. 1-4C is associated with (e.g., attached to) the body 105 of the radiation shield 101. The engagement member 157 is located remotely from the opening 141 so that a user may apply the detachment force to the engagement member without placing his or her hand or fingers close to the opening. For example, the engagement member 157 may be spaced from the longitudinal axis 151 of the cavity 107/ syringe barrel 109 to reduce the potential for radiation exposure. More particularly, the engagement member 157 may be located outside the zone Z2 including an axial projection of the opening 141 along the longitudinal axis 151. Still more particularly, the engagement member 157 may be located outside the zone Z1 from which there is a direct line of sight to at least a portion of the syringe barrel 109. Similarly, the engagement member 157 may be located in radially opposed relation with a portion of the shielding body 105 (e.g., alongside the body 105 of the radiation shield 101 on the exterior 113 of the cavity 107, as shown in FIG. 3). Moreover, the engagement member 157 may be positioned so that the body 105 of the radiation shield is between the engagement member and the syringe barrel 109. Some of the foregoing features are inclusive of others. It is also understood from the foregoing, that each of these features represents an improvement from the standpoint of radiation exposure than the prior art practice in which a person removed a needle cap by placing his or her fingers right at the opening of the radiation shield to pull the needle cap off the syringe.

The particular needle cap ejector 155 illustrated in FIGS. 1-4C comprises a leaf spring 161 secured at one end to the exterior sidewall of the radiation shield 101 formed by the radiation shielding body 105. In this embodiment, the engagement member 157 comprises a segment of the leaf spring 161 generally near the apex of a loop portion 167 of the leaf spring. The engagement surface is the radially outwardly facing surface of the engagement member 157 (which is the upper surface when oriented as in FIG. 4A). Other locations of the engagement surface that are remote from the opening 141 (e.g., outside the zones Z1 and Z2) are within the scope of the invention. The spring 161 extends from the exterior 113 of the cavity 107 into the forward end of the cavity through a small hole 163 in the radiation shielding body 105. The free end 165 of the spring 161 is positioned near the needle cap 125 when the needle cap is attached to the syringe 103 in the cavity 107. The hole 163 may be relatively narrow to reduce the potential for radiation to escape the radiation shield 101 through the opening. The hole 163 may also be configured as a slot oriented so that it is generally oblique with respect to the longitudinal axis 151 of the cavity 107/syringe barrel 109, which may also limit the escape of radiation from the radiation shield 101 through the slot. As shown in FIG. 4A, for example, the hole 163 may be configured and arranged relative to the cavity 107 so that there is substantially no direct line of sight from the exterior 113 of the cavity to the syringe barrel 109 through the hole 163. In one particular embodiment, for instance, the hole 163 for the spring 161 may be located at the front end of the radiation shield 101 and extend from the exterior 113 of the radiation shield inward toward the front end opening 141 obliquely generally along an axis oriented to form an acute angle A1 (e.g., in the range of about 10 to about 60 degrees) with the longitudinal axis 151 of the cavity 107/syringe barrel 109.

The spring 161 is biased to a first configuration (FIG. 4A) in which needle cap 125 may remain attached to the syringe 103. For example, the free end 165 of the spring 161 may be positioned adjacent the needle cap 125 in the first configuration, as shown in FIG. 4A. The free end 165 of the spring 161 is positioned adjacent the axially-facing surface 131 of the needle cap 125. Although the spring 161 does not engage the needle cap 125 in the first configuration of the illustrated embodiment, it is recognized that it possible for the free end 165 of the spring to contact the needle cap 125 in the first configuration without departing from the scope of the invention. In the first configuration the spring 161 may initially extend radially away from the body 105 at its connection thereto and then back radially inward into the hole 163, thereby forming the loop portion 167 of the spring.

The spring 161 is resiliently deformable by application of the detachment force to the engagement surface 157 to a second configuration (shown FIG. 4C) different from the first configuration. In the illustrated embodiment, for example, the detachment force may be directed radially inward (e.g., by squeezing the engagement member 157 of the spring 161 against the side of the shielding body 105 as shown in FIG. 4B) to flatten the loop portion 167 of the spring, at least to some degree, against the shielding body. It is noted that where the user engages the engagement member 157 (e.g., where his or her thumb contacts the engagement member) generally defines the engagement surface in this embodiment. The flattening of the loop portion 167 of the spring 161 causes the free end 165 of the spring to move farther out from the hole 163 in the side of the body 105 and through the opening 141 at the front of the radiation shield 101. The spring 161 engages the needle cap 125 and detaches it from the syringe 103 as the spring moves from its first configuration to its second configuration. For example, the spring 161 may be arranged so that its free end 165 engages the axially facing surface 131 of the needle cap 125 and pushes the needle cap away from the syringe 103 in a direction off of the needle 121 as the spring moves from its first configuration to its second configuration.

In one embodiment of a method of using the radiation shield 101 according to the present invention, an empty syringe 103 is placed in the cavity 107 of the radiation shield. The radioactive material (e.g., a radiopharmaceutical) is aspirated into the syringe 103 by inserting the tip of the needle 121 in to a reservoir (not shown) of the radioactive material and then pulling the plunger 147 toward the rear of the barrel 109. When a desired amount of the radioactive material is contained in the syringe barrel 109, the needle cap 125 is attached to the syringe 103 to enclose the tip of the needle 121 in the protective sheath of the needle cap 125. When it is time to use the syringe 103 to deliver the radioactive material contained therein to a destination (e.g., another container or a patient) a person detaches the needle cap 125 from the syringe by applying the detachment force to the engagement member 157 of the needle cap actuator 155. A person may hold the radiation shield 101 in one hand and use that same hand to apply the detachment force to the engagement member 157.

As shown in the embodiment depicted in FIGS. 4A-4C, for example, a person may hold the radiation shield 101 so that his or her thumb is adjacent the leaf spring 161. The person may then use his or her thumb to press the loop portion 167 of the leaf spring 161 radially inward toward the shielding body 105. This action flattens the loop portion 167 of the leaf spring 161, moving it from its first configuration toward its second configuration. By deforming the leaf spring 161 in this manner, the user causes the free end 165 of the leaf spring 161 to extend farther out of the hole 163 in the side of the shielding body 105 and move toward the needle cap 125. The free end 165 of the leaf spring 161 preferably engages the flange 133 at the open end of the needle cap 125 and still more preferably engages the axially facing surface 131 (as shown in FIG. 4B). As the leaf spring 161 is deformed farther toward its second configuration, the free end 165 of the leaf spring 161 may detach the needle cap 125 from the syringe 103 by pushing the needle cap away from the syringe barrel 109, as shown in FIG. 4C, for example. The needle cap 125 can drop off the needle 121 once released by the needle cap ejector 155.

By using the needle cap ejector 155 to detach the needle cap 125 in this manner, the user is able to detach the needle cap from a location that is remote from the opening 141 at the front of the radiation shield 101. Likewise, the person is preferably able to detach the needle cap 125 from the syringe 103 without placing his or her hands in the zone Z2 including all points adjacent the opening 141 and within an axial projection of the opening along the longitudinal axis 151 of the cavity 107/syringe barrel 109, and more preferably without placing his or her hands in the zone Z1 from which there is a line of sight to at least a part of the syringe barrel 109 through the opening 141, and still more preferably from a location in radially opposed relation with the body 105 of the radiation shield 101 during removal of the cap. Likewise, the body 105 of the radiation shield 101 may be positioned between the users hands and the syringe barrel 109 during removal of the needle cap 125. After removal of the needle cap 125, the syringe 103 may be used to deliver the contents of the syringe barrel 109 to a patient, to another container, any another destination in the same manner as a conventional syringe.

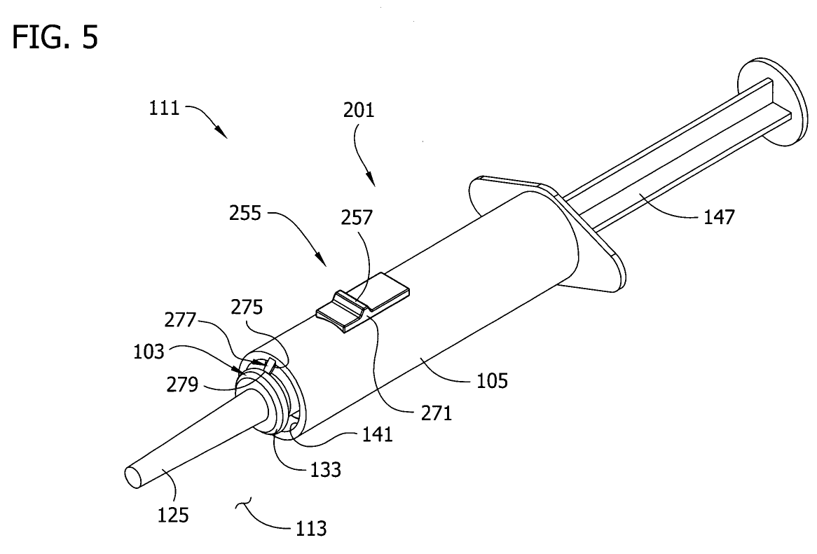

Another embodiment of a radiation shield 201 of the present invention is shown in FIGS. 5-7. Except as noted, this radiation shield 201 is constructed and operated in substantially the same way as the radiation shield 101 described above. The radiation shield 201 includes a slide button actuated needle cap ejector 255. The slide button 271 (broadly an “engagement member” and an “actuator”) is positioned alongside the body 105 on the exterior of the radiation shield 201. The slide button 271 defines an engagement surface 257 to which a detachment force may be applied to detach the needle cap 125 from the syringe 103, as described in more detail below. The engagement surface 257 is located remotely from the opening 141 at the front of the radiation shield. For example, the engagement surface 257 may be spaced from the longitudinal axis 151 of the cavity 107/syringe barrel 109 to reduce the potential for radiation exposure. More particularly, the engagement surface 257 may be located outside the zone Z2 including the points adjacent the opening 141 and within an axial projection of the opening. Still more particularly, the engagement surface 257 may be located outside the zone Z1 from which there is a direct line of sight to at least a portion of the syringe barrel 109. Similarly, the engagement surface 257 may be located in radially opposed relation with a portion of the shielding body 105 (e.g., adjacent the side of the shielding body on the exterior 113 of the cavity 107, as shown in FIG. 5).

There is a longitudinally extending slot 273 in the body 105 of the radiation shield 201. Moreover, the slot 273 opens into a longitudinally extending groove 275 in the inner surface of the body 105 of the radiation shield 201. The groove 275 extends from the slot 273 to the front end of the radiation shield 201. A needle cap detachment arm 277 is slidably mounted in the groove 275 and connected to the slide button 271 through the slot 273 (e.g. with any suitable fasteners, adhesives, welds, or the like (not shown)) so that the slide button and arm move together as a unit.

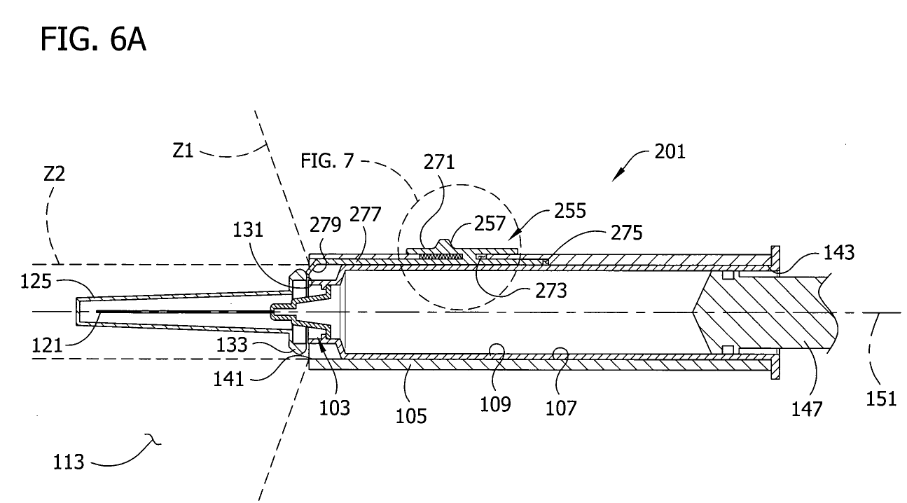

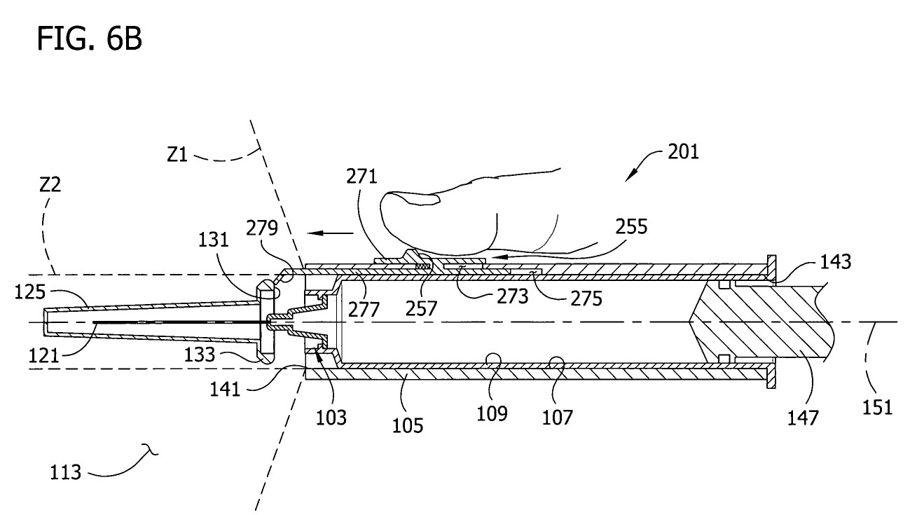

The slide button 271 and arm 277 are moveable longitudinally along the slot 273 from a first position (FIG. 6A) in which a needle cap 125 may remain attached to the syringe 103 forward to a second position (FIG. 6B). The arm 277 is positioned and arranged so that the arm may detach the needle cap 125 from the syringe 103 as the slide button 271 and arm are moved from their first position to their second position. The front end 279 of the arm 277 extends radially inward as shown in FIGS. 6A-6B to facilitate engagement of the needle cap 125 by the arm. For example, the front end 279 of the arm 277 is adapted to engage the needle cap 125 (e.g., the axially facing surface 131 on the open end of the needle cap) even if the largest diameter of the needle cap 125 is smaller than the diameter of the syringe barrel 109. Whether or not the front end of the arm extends radially inward, the type of needle cap used may also be selected to facilitate engagement of the needle cap by the arm. For example, a larger diameter needle cap (not shown) may be used so that the needle cap (e.g., a flange at the open end of the needle cap) extends radially outward a sufficient amount to facilitate contact with the arm. In view of the foregoing, those skilled in the art will be able to adapt the front end of the arm and/or the needle cap so that the arm contacts a desired portion of the needle cap as it moves from its first position toward its second position.

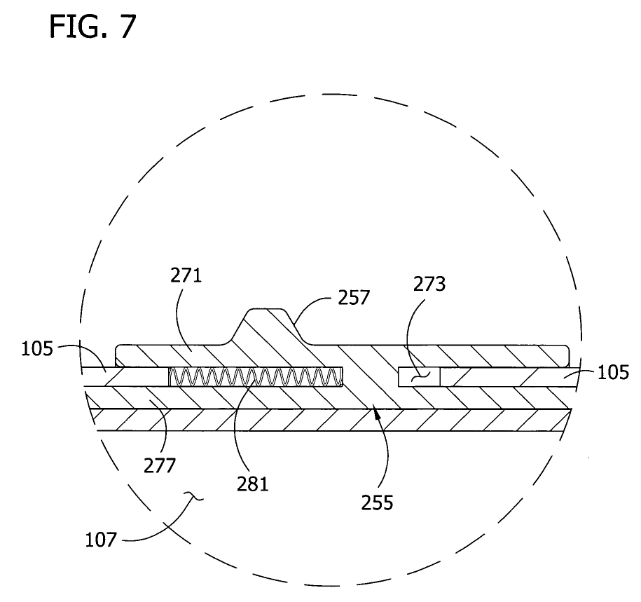

The slide button 271 and arm 277 may also be biased toward their first position. As shown in FIG. 7, for example, a biasing member (e.g., a spring 281 positioned to be compressed in the slot 273 when the slide button 271 moves forward) may be positioned in the slot to bias the slide button and arm to move toward their first position. The biasing force may be selected so the biasing member 281 has enough power to drive the slide button 271 and arm 277 back to their first position after a user slides them from their first position toward their second position.

The slide button needle cap ejector 255 may be constructed to prevent escape of radiation emitted in the cavity 107 through the slot 273. As shown in FIG. 7, for example, the slide button 271 and/or the needle cap detachment arm 277 may be configured so that they cover the slot 273 continuously as they move back and forth between their first and second positions. Preferably the slide button 271 and/or the needle cap detachment arm 277 are configured to cover the slot 273 continuously as they are moved from as far back in the slot 273 as they can be positioned to as far forward in the slot as they can be positioned. The slide button 271 and/or the needle cap detachment arm 277 may be constructed of a suitable radiation shielding material to prevent radiation emitted in the cavity 107 from escaping the radiation shield 201 through the slot 273.

During operation of the radiation shield 201, a user applies the detachment force to the engagement member 257 of the slide button 271 to move the slide button and detachment arm 277 forward toward their second position. As the detachment arm 277 moves forward, it detaches the needle cap 125 from the syringe 103 by pushing the needle cap away from the syringe barrel 109 in a direction off of the needle 121. Although the arm 277 and needle cap 125 are depicted in FIG. 6A as already being in contact when the arm is in the first position, it is recognized that the arm might not engage the needle cap until after the arm begins movement toward its second position. As the needle cap 125 is detached, the user is able to keep his or her hands away from the areas (e.g., zones Z1, Z2) where radiation exposure is a greater concern (in the same manner as set forth for the radiation shield 101 described above). Further, the slide button 271 and/or the needle cap detachment arm 277 cover the slot 273 and thereby protect the user from radiation that might otherwise escape the radiation shield 201 through the slot. After the needle cap 125 is detached from the syringe 103, the user may release the slide button 271. Release of the slide button 271 allows the biasing member 281 to automatically move the slide button 271 and the needle cap detachment arm 277 back to their first position. After removal of the needle cap 125, the syringe 103 can be used to deliver the contents of the syringe barrel to a patient, another container, or another destination.

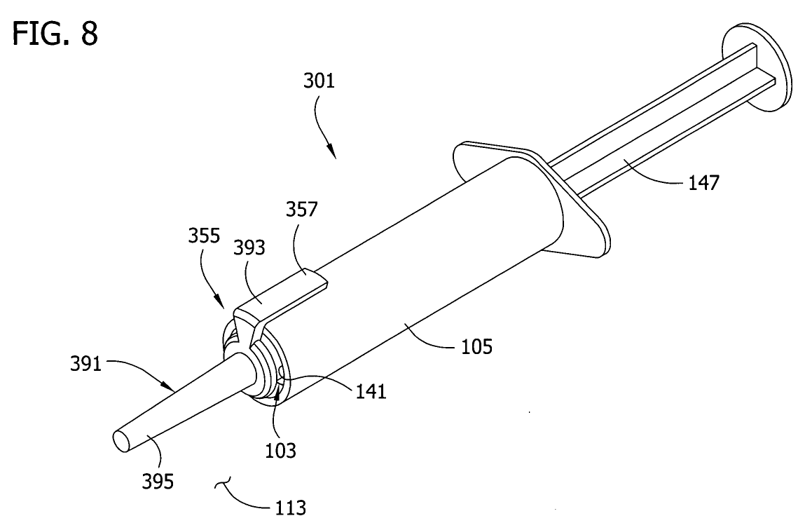

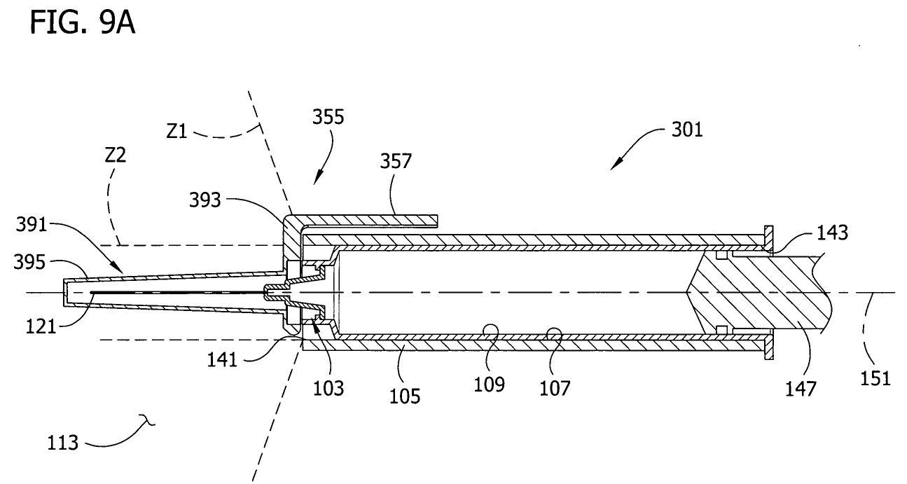

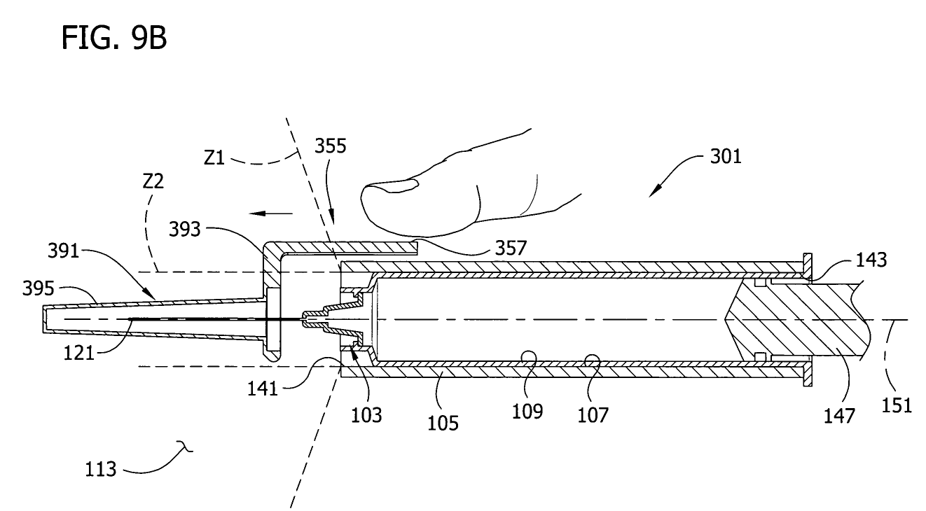

Yet another embodiment of the invention is shown in FIGS. 8-9B. In this embodiment, a conventional radiopharmaceutical syringe 103 is loaded into a conventional radiation shield 301. A needle cap 391 is attached to the syringe 103 in the same manner as the needle cap 125 described above. However, the needle cap 391 includes a needle cap ejector 355 comprising an arm 393 that is secured to a tubular body 395 of the needle cap. The arm 393 extends from the body 395 of the needle cap to a location that is remote from the opening 141 at the front of the radiation shield 301. For example, the arm 393 may extend to a location that is outside the zone Z2 that includes the points adjacent the opening 141 and within an axial projection of the opening along the axis of the cavity 107/syringe barrel 109, and more preferably to a location that is outside the zone Z1 that includes the points having a direct line of sight to at least part of the syringe barrel through the opening 141, and still more preferably to a location in radially opposed relation with (e.g., alongside) the body 105 of the radiation shield 301. As shown in FIGS. 8-9B, for example, the arm 393 is shaped and sized so that it may be secured to the tubular body 395 of the needle cap at its open end and may extend from the open end of the needle cap 391 around the front edge of the radiation shield 301 and then rearward along and adjacent the side of the body 105 of the radiation shield.

The arm 393 may be integrally formed with the body 395 of the needle cap 391 (as shown). Alternatively, the arm 393 may be bonded (or otherwise secured) to the tubular body 395 of an ordinary needle cap after manufacture of the needle cap.

The portion of the arm 393 that is remote from the opening 141 may be positioned and arranged so that a user may apply the detachment force to an engagement member 357 of the arm to detach the needle cap 391 from the syringe 103 from a position that is remote from the opening. In this embodiment, a rearward segment of the arm 393 defines the engagement member 357. As shown in FIG. 9B, for example, the remote portion of the arm 393 may be positioned and arranged so a user may push the engagement member 357 of the arm with a thumb or finger of one hand while holding the radiation shield 301 with the same hand.

In any case, during use a user applies the detachment force to the engagement member 357 of the arm 393, thereby pushing the arm toward the front end of the radiation shield 301. The detachment force is transferred through the arm 393 to the body 395 of the needle cap 391, which is thereby detached from the syringe 301. All the while, the user is able to keep his or her hand out of the areas where radiation exposure is of greater concern. In particular, the user may remove the needle cap 391 while keeping his or her hands remote from the opening 141, preferably while keeping his or her hands spaced radially from the longitudinal axis 151 of the cavity 107/syringe barrel 109, and more preferably while keeping his or her hands out of the zone Z2 including the points adjacent the opening and within an axial projection of the opening along the longitudinal axis 151, and still more preferably while keeping his or her hands out of the zone Z1 including the points having a direct line of sight to a part of the syringe barrel 109 through the opening 141, and still more preferably while keeping his or her hand in radially opposed relation with the body 105 of the radiation shield 301. The user may maintain his or her hand in a position so that the body 105 of the radiation shield 301 is between the user's hand and the radioactive material in the syringe barrel 109. After the needle cap 391 is removed, the syringe 103 may be used to deliver the contents of the syringe barrel to a patient, another container, or another destination.

When introducing elements of the present invention or the preferred embodiments thereof, the articles “a”, “an”, “the”, and “said” are intended to mean that there are one or more of the elements. The term “comprising”, “including”, and “having” are intended to be inclusive and mean that there may be additional elements other than the listed elements.

As various changes could be made in the above constructions and methods without departing from the scope of the invention, it is intended that all matter contained in the above description and shown in the accompanying figures shall be interpreted as illustrative and not in a limiting sense.

What is claimed is:

1. A radiation shielding assembly comprising:
a syringe comprising:
a barrel for containing a radioactive substance; and
a needle located at one end of the barrel;
a radiation shielding body having a cavity defined therein for receiving at least a portion of the barrel of the syringe, the body having at least one opening into the cavity defined therein for delivery of a radiopharmaceutical in the barrel of the syringe exterior of the cavity through the opening; and
a needle cap ejector supported by the radiation shielding body, the needle cap ejector being operable to detach the needle cap from the syringe upon manual application of a detachment force to the needle cap ejector.

2. The assembly of claim 1, wherein the needle cap ejector comprises an engagement member having an engagement surface located remotely from the opening, the needle cap ejector being operable to detach the needle cap from the syringe upon application of the detachment force to the engagement surface.

3. The assembly of claim 2, wherein a zone of radiation exposure is defined by all locations within an axial projection of the opening away from the radiation shielding body, the engagement surface being located exterior of the cavity and outside the first zone.

4. The assembly of claim 3, wherein the engagement surface is in radially opposed relation with at least a portion of the radiation shielding body.

5. The assembly of claim 4, wherein the engagement surface is located adjacent the radiation shielding body.

6. The assembly of claim 1, wherein the needle cap ejector comprises a spring biasing the needle cap ejector to a first position in which the needle cap may remain attached to the syringe, the spring being resiliently deformable to a second position different from the first for detaching the needle cap from the syringe.

7. The assembly of claim 6, wherein a portion of the spring defines the engagement member and the engagement surface, the spring being connected at one end to the radiation shielding body and having a free end positioned at the opening, the spring being biased to a first configuration in the first position of the needle cap ejector, the spring being resiliently deformable by application of the detachment force to the engagement surface to a second configuration in the second position of the needle cap ejector.

8. The assembly of claim 6, wherein the radiation shielding body has a hole defined therein, the spring being connected to an exterior of the shielding body and extending through the hole.

9. The assembly of claim 8, wherein the cavity and the hole each have a longitudinal axis, the longitudinal axis of the hole being inclined relative to the longitudinal axis of the cavity.

10. The assembly of claim 1, wherein the engagement member comprises an actuator slidably mounted on the radiation shielding body.

11. the assembly of claim 10, further comprising a biasing member that biases the actuator toward a first position, the actuator being manually moveable against the bias of the biasing member to a second position for detaching the needle cap from the syringe.

12. The assembly of claim 10, wherein the actuator is made of a radiation shielding material.

13. The assembly of claim 1, wherein the needle cap ejector and radiation shielding body are shaped and arranged so that a person can hold the shielding body and apply the detachment force to the engagement surface with a single hand.

14. A method of using a radiation shielding assembly comprising a syringe and a radiation shield disposed about at least a portion of the syringe, the method comprising:
exerting a force on a needle cap ejector of the assembly, wherein radiation shielding material of the radiation shield is located between the ejector and a barrel of the syringe during the exerting; and
detaching a needle cap from a needle of the syringe due to the exerting.

15. The method of claim 14, wherein the force comprises a force vector that is non-parallel with a longitudinal reference axis of the syringe.

16. The method of claim 14, wherein the force comprises a force vector that is substantially perpendicular to a longitudinal reference axis of the syringe.

17. The method of claim 14, wherein the force comprises a force vector that is substantially parallel to a longitudinal reference axis of the syringe.

18. The method of claim 14, further comprising transferring at least a portion of the force to the needle cap.

19. The method of claim 14, wherein the ejector is integral with the needle cap.

20. The method of claim 14, wherein the detaching comprises contacting only a portion of an axially facing surface of the needle cap with the ejector.
